(19) United States
(12) Patent Application Publication
(10) Pub. No.: US 2004/0185461 A1
(43) Pub. Date: Sep. 23, 2004

(54) Oligonucleotides for detecting hybridisation

(76) Inventors: Mikhail Sergeevich Shchepinov, Oxford (GB); Edwin Mellor Southern, Oxford (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/479,887

(22) PCT Filed: Jun. 7, 2002
(86) PCT No.: PCT/GB02/02596

(30) Foreign Application Priority Data
Jun. 8, 2001 (GB) 0114007.8

(51) Int. Cl. C12Q 1/68; C07H 21/04

(52) U.S. Cl. 435/6; 536/24.3

(57) ABSTRACT

A polynucleotide comprises a central region and two terminal regions which are complementary with each other and are capable of undergoing hybridisation to form a stem loop structure, wherein the terminal regions are in the reverse orientation to that of the central region. The polynucleotide may be labelled with a fluorophore at one end and a quenching molecule at the other and used in methods to detect hybridisation between a target polynucleotide and the central region.

### FIELD OF THE INVENTION

This invention relates to oligonucleotides and their use, in particular to their use in detecting hybridisation events.

### BACKGROUND TO THE INVENTION

Polynucleotide arrays are now an established technology, useful in a wide variety of assay procedures. Typically, the arrays comprise a plurality of distinct sites, each comprising a high density of single-stranded polynucleotides immobilised by a covalent linkage to a planar solid support material. The arrays may be fabricated by, for example, contact printing techniques.

The arrays are particularly suitable for DNA sequencing procedures, for genotyping or for the detection of genetic mutations. Many techniques are hybridisation based. That is, they rely on the detection of hybridisation between an immobilised polynucleotide of the array and a complementary target polynucleotide. If the sequence of the immobilised polynucleotide is known, then the detection of hybridisation will reveal the sequence of the target. This is particularly useful in many genotyping experiments or for the detection of genetic mutations. The detection of the hybridisation events is therefore an important aspect in the success of the method.

Detection of hybridisation is usually achieved using fluorescent labels. For example, the target polynucleotide may be fluorescently labelled. Hybridisation events may then be detected by measuring the fluorescence on the array, after first removing any non-hybridised target polynucleotides. A disadvantage of this technique is that it is not always possible to label all the target polynucleotides in a sample.

A recent development has been the introduction of “molecular beacons”, which are oligonucleotides having complementary terminal regions capable of forming stem-loop structures. The oligonucleotides are labelled at one end with a fluorophore, and at the other end with a quenching group. In the stem-loop configuration, the fluorescence is quenched due to the close positioning of the two groups. However, when contacted with a suitable target polynucleotide that has a sequence complementary to a part of the oligonucleotide, the stem-loop configuration becomes disrupted and the target polynucleotide hybridises to its complement. This separates the labelling groups, permitting fluorescence to be detected. This system may be used with oligonucleotide arrays on solid supports, but the stem-loop structures can also be used in solution. A review of the molecular beacons system is disclosed in Tyagi et al., Nature Biotechnology, 1996; 14: 303-308. This is generally illustrated in FIG. 1a. While the molecular beacons approach has many advantages, a problem exists that it is possible for a false positive signal to be generated by only partial hybridisation of the target to one end of the stem-loop structure. This is illustrated in the right-hand part FIG. 1b. This problem remains even if the stem parts are very short.

### SUMMARY OF THE INVENTION

The present invention is based, in part, on the realization that the detection of hybridisation-based events can be improved by providing stem-loop structures, in which the terminal regions (stems) cannot hybridise with any target polynucleotide that partially hybridises with any other part of the structure.

According to a first aspect of the invention, a polynucleotide comprises a central region and two terminal regions that are complementary with each other and are capable of undergoing hybridisation to form a stem-loop structure, wherein the terminal regions are in the reverse orientation to that of the central region. As will be understood by the skilled person, the term “orientation” refers to the direction in which polynucleotides are said to be positioned, with regard to the 3′ and 5′ termini.

By having the terminal regions in the reverse orientation, it becomes impossible for the terminal regions to cross-hybridise with a target polynucleotide that has partially hybridised with the central region. The polynucleotides may therefore be used in hybridisation-based detection assays, resulting in significant reduction in false positive results.

According to a second aspect of the invention, a method for detecting the hybridisation of a target polynucleotide to its complement comprises contacting the target polynucleotide under hybridising conditions, preferably highly stringent conditions, with a polynucleotide as defined above, where the complement is, or is a part of, the central region, and which comprises a fluorescent moiety attached at one terminal region and a quenching group attached at the other terminal region, and detecting fluorescence.

In the absence of hybridisation with the target polynucleotide, the stem-loop structure is retained, and no fluorescent signal is generated, due to the quenching effect. On hybridisation, the stem-loop structure is disrupted, and a fluorescent signal can be detected. This is generally illustrated in FIG. 2a. In contrast to the conventional molecular beacons approach, the use of stem structures in the reverse orientation to that of the loop region, reduce the likelihood of false positive results due to cross-hybridisation between the target and a stem structure. Partial hybridisation and its failure to give a false positive results is shown in the right-hand part of FIG. 2b.

### BRIEF DESCRIPTION OF THE DRAWINGS

FIG. 1 illustrates conventional stem-loop structures used as molecular beacons and positive and false positive identifications wherein □ represents a fluorophore and ▪ represents a quencher molecule; and

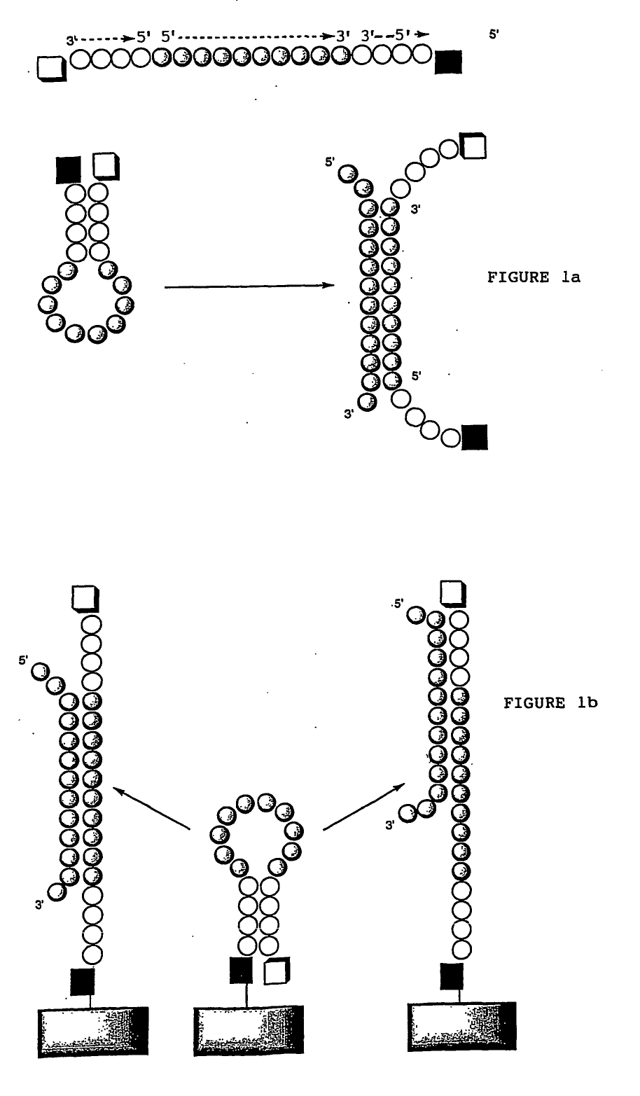

FIG. 2 illustrates the stem-loop structures of the present invention, where the complementary stems are in the opposite orientation to that of the loop region, and positive identification and prevention of false positive identifications.

### DESCRIPTION OF THE INVENTION

The present invention relates to improvements in the detection of hybridisation reactions, in particular those occurring on polynucleotide arrays. The invention can also be used for detecting hybridisation in solution systems. The application of the invention in solution systems has the added advantage that it allows “real-time” measurements to be obtained, to follow the course of a reaction. The polynucleotides of the invention may also be used in living cells.

The polynucleotide stem-loop structures of the invention are designed so that the polynucleotides that form the stems are in the reverse orientation (inverted) with respect to that of the loop region.

Thus, for example, if the loop region comprises DNA in the 5′ to 3′ orientation, the DNA that forms the stems is in the 3′ to 5′ orientation. This ensures that, under the appropriate reaction conditions, any polynucleotide that only partially hybridises to the loop region will be unable also to hybridise with the stems as it will not be in the correct orientation, and will therefore not form a stable duplex under hybridisation conditions. The partially hybridised polynucleotide therefore does not result in a false positive signal.

The reference to the 5′ and 3′ orientation of nucleic acids has its conventional meaning and is well understood in the art.

Typically, the stem-loop structure comprises one single polynucleotide molecule. However, it is possible for the loop region and stem regions to be separated by linker molecules.

The loop region will be, or will comprise, that region intended for hybridising to a target polynucleotide, i.e. the complement of the target polynucleotide.

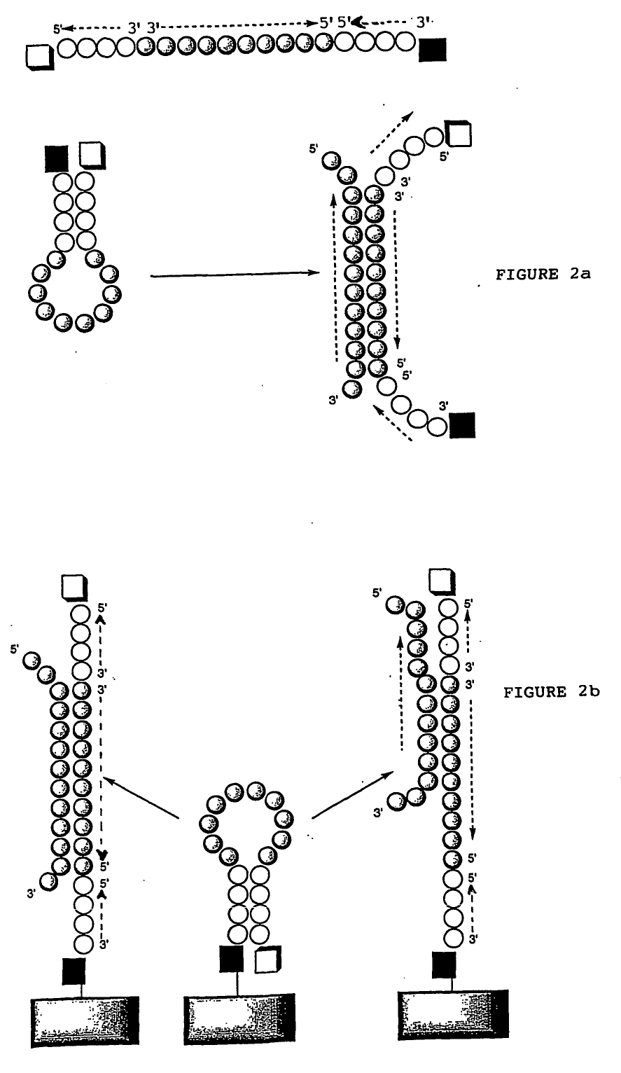

The polynucleotides may be produced using conventional synthetic approaches, e.g. using phosphoramidite chemistry. Achieving the linkage of the different regions can be carried out by appropriate placing of the blocking groups used during the synthesis procedure. For example, in attaching two 3′ terminal regions (see FIG. 2), it will be necessary to include blocking groups at the 5′ ends. Suitable methods will be apparent to the skilled person.

The stems will be labelled with a fluorophore on one stem and a quenching group on the other. This ensures that, while the polynucleotide is in the stem-loop configuration, fluorescence does not occur, or occurs only to a very low level. When the stem-loop configuration is disrupted, the fluorophore and quenching group are no longer in proximity, and fluorescence can occur. The two groups are preferably located at the terminal ends of the stems, but may be located elsewhere along the stems. The two groups should be attached to the polynucleotide in such a way and in such positions that they are in close proximity when the stem-loop structure is formed. This ensures that the quenching effect occurs.

Suitable fluorophores and quenching groups are known in the art, and include those used in conventional molecular beacons assays. For example, 5-(2′aminoethyl)aminoaphthalene-1-sulphonic acid (EDANS) is a suitable fluorophore, and 4-(4′dimethylaminophenylazo) benzoic acid (DABCYL) is a suitable quenching group. Other groups are known and can be chosen so that a quenching effect can be achieved. Linking the groups to the stems can be achieved using known techniques.

The regions of the polynucleotide (terminal regions) that form the stems will typically comprise from 2 to 20 nucleic acids, preferably 3 to 10 nucleic acids, more preferably 4 to 8 nucleic acids, often 6 or less and most preferably 4 nucleic acids. The number of nucleic acids should be chosen so as to ensure that adequate hybridisation to form the stem-loop structure is achieved, but that appropriate conditions can be used to disrupt the structure, permitting hybridisation with a target polynucleotide to occur.

The central region may be of any suitable size, sufficient to allow hybridisation of a complementary target polynucleotide. Typically, the central region will comprise at least 10 nucleic acids, preferably more than 15 nucleic acids and more preferably more than 20 nucleic acids. That part of the central region that hybridises with the target is preferably larger than the stems, so that a more stable duplex is formed.

The polynucleotides may be used in a polynucleotide array, i.e. a plurality of polynucleotides that are located in distinct areas on a solid support surface. The polynucleotides may be DNA or RNA, or synthetic derivatives thereof. Polynucleotide arrays are now well known in the prior art, and their manufacture will be appreciated by the skilled person in the art. For example, see U.S. Pat. No. 5,744,305. The polynucleotides will usually be attached to the solid support surface through a covalent linkage, although the use of non-covalent linkages is also within the scope of the invention. Suitable surface chemistries which may be used to link the polynucleotides to the array will be apparent to the skilled person, and include amide, epoxide or silane-based chemical linkages. The solid support may be made from any conventional material, including silicon, glass, ceramics or plastics. The support will typically have a surface area of about 1 cm² although larger surface areas are also within the scope of the present invention. The solid support will usually comprise greater than 1000 of the polynucleotides that form the stem-loop structures. Higher densities are also desirable, and the solid support may comprise from 10³-10¹⁰ polynucleotides per cm², preferably 10⁷-10⁹ polynucleotides per cm². The polynucleotides may be the same or different. The polynucleotides may be labelled with the same or different fluorophores and quenching groups.

The polynucleotides will usually be attached to the solid support at one terminus, leaving the remaining polynucleotide exposed for duplex formation with a suitable complementary polynucleotide (target polynucleotide).

The polynucleotides may be used in a method for detecting an hybridisation event. The method comprises contacting a polynucleotide of the invention with a sample comprising a target polynucleotide, preferably under stringent hybridising conditions, and detecting fluorescence.

Conditions for carrying out the hybridising reaction will be apparent to the skilled person, and variations in buffer, salt content, temperature and target polynucleotide concentration will be apparent from conventional hybridising reactions. It will be apparent that the conditions must be chosen so that, in the absence of hybridisation with a target polynucleotide, the stem-loop configuration is maintained. Therefore, a temperature above the melting temperature of the stem duplex should not ordinarily be used, as otherwise the stem-loop configuration will be disrupted and a fluorescence signal generated in the absence of hybridisation. Preferably highly stringent hybridising conditions are used. Stringent hybridising conditions are known to the skilled person, and are chosen to reduce the possibility of non-complementary hybridisation. Examples of suitable conditions are disclosed in Nucleic Acid Hybridisation: A Practical Approach (B. D. Hames and S. J. Higgins, editors IRL Press, 1985).

In the method, suitable washing steps may be applied after hybridisation to remove partially hybridised polynucleotides.

The method can be carried out in homogeneous solution, and in living cells.

The detection of fluorescence ay be carried out by conventional microscopy-based techniques. For example, confocal microscopy using a CCD camera may be used to monitor fluorescence. Conventional detection systems include those currently used in the known molecular beacon approach.

The target polynucleotide may be derived from a biological sample, or may be made synthetically. The target polynucleotide may be derived from a patient's genome, in a genotyping experiment or in the study of single nucleotide polymorphisms (SNPs).

1. A polynucleotide comprising a central region and two terminal regions which are complementary with each other and capable of undergoing hybridisation to form a stem loop structure, wherein the terminal regions are in the reverse orientation to that of the central region.

2. A polynucleotide according to claim 1, wherein one terminal region is labelled with a fluorophore and the other is labelled with a quenching group.

3. A polynucleotide according to claim 2, wherein the fluorophore is fluorescein.

4. A polynucleotide according to any preceding claim, wherein the terminal regions comprise from 3 to 10 nucleic acids.

5. A polynucleotide according to any of claims 1 to 3, wherein the terminal regions comprise 6 or less nucleic acids.

6. A polynucleotide according to any preceding claim, wherein the terminal regions comprise 4 nucleic acids.

7. A polynucleotide according to any preceding claim, immobilised on a solid support.

8. A solid support material having thereon at least one immobilised polynucleotide according to any preceding claim.

9. A solid support according to claim 8, having immobilised thereon greater than 1000 of the said polynucleotides.

10. A method for detecting the hybridisation of a target polynucleotide to its complement, comprising:
contacting the target polynucleotide with a polynucleotide according to claim 2, or any claim pendant to claim 2, under stringent hybridising conditions and detecting fluorescence.

11. A method according to claim 10, wherein the target polynucleotide is immobiled on a solid support.
